(19) United States
(12) Patent Application Publication
(10) Pub. No.: US 2026/0276500 A1
(43) Pub. Date: Sep. 17, 2026

(54) METHOD FOR OBTAINING CIRCUMFERENTIAL MECHANICAL PROPERTY PARAMETERS OF PIPE

(71) Applicant: Shenyang Aircraft Corporation (SAC), Shenyang (CN)

(72) Inventors: Xin PAN, Shenyang (CN); Zhe LIU, Shenyang (CN); Weinan CHEN, Shenyang (CN); Minggang LIU, Shenyang (CN); Meiping LI, Shenyang (CN)

(21) Appl. No.: 19/371,930

(22) Filed: Oct. 28, 2025

Related U.S. Application Data
(63) Continuation of application No. PCT/CN2024/104699, filed on Jul. 10, 2024.

(30) Foreign Application Priority Data
Oct. 27, 2023 (CN) 202311401845.7

(51) Int. Cl. G01N 3/08; G01N 3/06

(52) U.S. Cl. CPC G01N 3/08; G01N 3/068; G01N 2203/0274

(57) ABSTRACT

The present invention provides a method for obtaining circumferential mechanical property parameters of a pipe, and belongs to the field of manufacturing engineering/digital information. The present invention adopts a flexible tensile test fixture to conduct a tensile test on a circumferential tensile test sample of a pipe. During the tensile test, it is necessary to use a non-contact video extensometer and a universal tensile testing machine to obtain tensile measurement data of the pipe; then the tensile measurement data of the pipe is preprocessed, true stress-true strain data of the circumferential tensile test sample of the pipe is obtained by calculation using the preprocessed data, and a strength coefficient and a strain hardening index in a plastic section are obtained by calculation through a method of linear regression fitting by setting starting and ending strains of the plastic section.

### CROSS REFERENCE TO RELATED APPLICATIONS

This application is a Continuation of co-pending International Application No. PCT/CN2024/104699, filed on Jul. 10, 2024, which claims priority of Application No. 202311401845.7 filed in China on Oct. 27, 2023, the entire contents of both of which are hereby incorporated by reference.

### TECHNICAL FIELD

The present invention belongs to the field of manufacturing engineering/digital information, and particularly relates to a method for obtaining circumferential mechanical property parameters of a pipe. A flexible tensile test fixture is provided to conduct a tensile test to pipes of various specifications, obtain circumferential mechanical test data of the pipes efficiently and accurately, and obtain circumferential mechanical property parameters of the pipes suitable for finite element simulation by calculation.

### BACKGROUND

A pipeline system is an important part of a power plant, and the safety and reliability of the pipeline system will directly affect the overall safety of a structure. Hydraulic, fuel, lubricating oil, environmental control, oxygen and other pipeline systems are widely distributed in a complex product structure. The pipeline systems have different shapes and intricate distribution, and play the roles of pressure transmission, fuel transmission, cable protection, etc. For example, high pressure pipeline fracture is a main cause of an engine failure.

Traditional methods such as fluid-structure coupling vibration and fatigue life failure analysis have the problems of complicated parts and difficult processing. Finite element analysis of pipeline system strength can provide a basis for system design and optimization. The accuracy of finite element analysis depends on the accuracy of material parameters. Relevant standards for axial mechanical property tests of pipes are provided, but no relevant standard for circumferential mechanical property tests is available at present. Therefore, it is necessary to obtain circumferential properties of a pipe quickly and accurately, and obtain parameters required for finite element analysis by calculation.

In a traditional mechanical property test, a strain gauge is usually used for measurement, the measuring range of which is very limited. A contact measurement method has a certain influence on a measured part, and each gauge needs to be calibrated separately. With the increase of test times, the measurement accuracy will also be affected by wear. In a destructive tensile test, it is impossible to track a stage of large deformation or fracture of a test sample, and data after removing the gauge is obtained by reckoning, so the accuracy is limited.

The above shortcomings can be effectively avoided by using a digital image correlation method to measure a deformation amount of the test sample. The method is based on a mode of tracking speckle features on a surface of a material, and tracking specific position features to measure strain data of the material. By conducting correlation matching to gray features around a point in a speckle pattern before and after deformation, the position of the point in different states during deformation can be tracked. The measurement mode is non-contact measurement, so the test sample will not be affected by any external force, the measurement accuracy is high, the measuring range is large, and tracking can be conducted until the test sample is completely destroyed to obtain accurate test data of the whole process.

An accurate and comprehensive description of a strain hardening behavior of the material requires the use of a true stress-strain curve, because stress and strain in an engineering stress-curve are measured by an original cross-sectional area and an original range length of a range section of the test sample, and do not represent actual instantaneous stress and strain. However, true stress-strain data cannot be directly provided by a circumferential property test, and stress-strain data used for finite element simulation needs to be obtained by calculation after data processing.

### SUMMARY

The present invention aims to solve the problems in the prior art, and proposes a method for obtaining circumferential mechanical property parameters of a pipe.

The technical solution of the present invention is as follows:

A method for obtaining circumferential mechanical property parameters of a pipe, comprising the following steps:
Step (1): adopting a flexible tensile test fixture used for testing circumferential mechanical property parameters of a pipe to conduct a tensile test on a circumferential tensile test sample of the pipe, and making the circumferential tensile test sample subjected to pulling forces until fracture occurs; during the tensile test, a deformation amount in a whole tension process of the circumferential tensile test sample is obtained in real time based on a digital image correlation method by using a non-contact video extensometer, thus to obtain an engineering strain of the circumferential tensile test sample, and then obtain time of the tensile test and a force of the tensile test in combination with a universal tensile testing machine; the deformation amount of the circumferential tensile test sample, the engineering strain of the circumferential tensile test sample, the time of the tensile test and the force of the tensile test are collectively referred to as tensile measurement data of the pipe;
Step (2): preprocessing the tensile measurement data of the pipe; preprocessing means sequentially conducting: all abnormal data clearing before starting sampling, force check and convert, data cleaning and data screening; the preprocessed data includes only two types of data: the force of the tensile test and the engineering strain of the circumferential tensile test sample;
Step (3): obtaining true stress-true strain data of the circumferential tensile test sample of the pipe by calculation according to the data obtained in step (2);
Step (4): obtaining a strength coefficient K and a strain hardening index β in a plastic section by calculation through a method of linear regression fitting by setting starting and ending strains of the plastic section according to the true stress-true strain data of the circumferential tensile test sample; the strength coefficient K and the strain hardening index β in the plastic section as well as the true stress-true strain data of the circumferential tensile test sample are used for finite element analysis of a pipeline system.

Further, the flexible tensile test fixture comprises an upper fixture and a lower fixture, and each fixture comprises a chuck, a D-shaped block and a pin;

The chuck comprises a clamping part, a gradual expansion part and a D-shaped block mounting part; the clamping part is a thin cylinder and is used for matching with a V-shaped clamping block of the universal tensile testing machine; upper and lower ends of the gradual expansion part are fixed with a bottom end of the clamping part and a top end of the D-shaped block mounting part, respectively; a D-shaped block mounting slot is formed in and penetrates through a side surface of the D-shaped block mounting part, and a length of a notch of the D-shaped block mounting slot is less than that of the circumferential tensile test sample; the circumferential tensile test sample is placed on an arc section of the D-shaped block; during the tensile test, the circumferential tensile test sample and the D-shaped block are placed in the notch of the D-shaped block mounting slot; the pin transversally penetrates through the D-shaped block mounting part of the chuck and side surfaces of the D-shaped block to connect the D-shaped block with the D-shaped block mounting part of the chuck, and the D-shaped block mounting part of the chuck, the D-shaped block and the pin are in clearance fit.

Further, the D-shaped block is a flexible clamping element, and a diameter of the D-shaped block is 1 mm-2 mm smaller than that of a circumferential tensile test sample to be measured; D-shaped blocks with different diameters are designed to realize circumferential tension of circumferential tensile test samples of pipes with different diameters.

Further, the circumferential tensile test sample is cut from a pipe to be measured, the circumferential tensile test sample has a symmetrical structure, and range sections of the circumferential tensile test sample are located on both sides of a middle part of the circumferential tensile test sample.

Further, it is assumed that the D-shaped block mounting slot has a length of B and a width of A, the pipe has a diameter of D, the circumferential tensile test sample has a width of L₀, then B>D/2+L₀, A is not less than L₀, thickness δ/diameter D of the pipe is less than 0.05, a length of each range section of the circumferential tensile test sample is L=π·D/12, and transition parts of the range sections are smoothed.

Further, the tensile test comprises the following steps:
1) Conducting speckle treatment to the range sections of the circumferential tensile test sample: first, spraying a white matte developer uniformly, and spraying black matte paint spots after natural air drying to form speckles;
2) Selecting matching D-shaped blocks according to a size of the circumferential tensile test sample, and clamping the circumferential tensile test sample on the flexible tensile test fixture; then clamping the flexible tensile test fixture on the universal tensile testing machine, and using polytetrafluoroethylene to lubricate the contact positions of the D-shaped blocks with the circumferential tensile test sample;
Placing the range sections after speckle treatment on side surfaces, making the range sections within a measuring range of the non-contact video extensometer, setting a range to ensure that the range sections are completely within a field of view of a high-speed camera of the non-contact video extensometer, adjusting a focal length and conducting range calibration;
3) Starting the universal tensile testing machine and the non-contact video extensometer to conduct the tensile test, and obtaining the tensile measurement data of the pipe.

Further, the specific process of step (2) is as follows:
Using the tensile measurement data of the pipe as an output data set, including the force F, the deformation amount ΔL, the engineering strain ε_{eng} and the time t; reading a data column of the force F and denoting as column A0=[A01,A02, . . . , A0n]T, reading a data column of the deformation amount ΔL and denoting as column B0=[B01,B02, . . . , B0n]T, reading a data column of the engineering strain ε_{eng} and denoting as column C0=[C01,C02, . . . , C0n]T, and reading a data column of the time t and denoting as column D0=[D01,D02, . . . , D0n]T;
(2.1) All abnormal data clearing before starting sampling;
Reading the data column D₀, D₀₍ₚ₊₁₎−D₀ₚ=Tₚ, and p∈{1,2,3,4, . . . , n−1}; Tₚ represents a time difference between row p+1 and row p;
When

$$0 < {T}_{p} < k = \frac{1}{f} ,$$

data of row p+1 is marked as normal; otherwise, row p+1 and row p are marked as abnormal, wherein 1.5<k<2, and f is a sampling frequency;
When 5 normal marks appears continuously, the reading is stopped, and all data in rows with abnormal marks is cleared; obtaining a new data set: denoting a data column of the force F as column A₁=[A₁₁,A₁₂, . . . , A₁ₘ]^{T}, denoting a data column of the deformation amount ΔL as column B₁=[B₁₁,B₁₂, . . . , B₁ₘ]^{T}, denoting a data column of the engineering strain ε_{eng} as column C₁=[C₁₁,C₁₂, . . . , C₁ₘ]^{T}, and denoting a data column of the time t as column D1=[D11,D12, . . . , D1m]T; m<n;
(2.2) Force check and convert;
Reading all data [A₁₁,A₁₂, . . . , A₁ₘ]^{T} in column A₁, and obtaining a maximum force Fmax;
If Fmax≤200, [A₁₁,A₁₂, . . . , A₁ₘ]^{T}×1000=[A′₁₁,A′₁₂, . . . , A′₁ₘ]^{T}, and [A₁₁,A₁₂, . . . , A₁ₘ]^{T} is overwritten with [A′₁₁,A′₁₂, . . . , A′₁ₘ]^{T} to obtain a new column A₁;
If Fmax>200, a next step is conducted directly;
(2.3) Data cleaning;
Reading all data [C₁₁,C₁₂, . . . , C₁ₘ]^{T} in column C₁, and judging whether C_{1q}≤0, wherein q∈{1,2,3, . . . , m}; recording the last position where C_{1q} is zero or negative, clearing all data [A₁,B₁,C₁,D₁] before the position, deleting the data column of the deformation amount ΔL and the data column of the time t, and finally forming a new data set [A₂,C₂];
(2.4) Data screening;
Conducting periodic sampling and data screening to the data set [A₂,C₂] to reduce data and reduce a calculation amount on the premise of ensuring that a true stress-true strain curve can transition smoothly.

Further, the data screening is conducted in the following 3 screening modes:
Mode I: sampling at fixed interval;
Specifying a single value as an interval for periodic sampling, conducting sampling at fixed interval to the data set [A₂,C₂], sequencing the sampled data, and denoting the data as [A₃,C₃], wherein A₃ is a data set of the force F, and C₃ is a data set of the engineering strain ε_{eng};
Mode II: sampling at variable interval;
Calculating a sampling interval according to a total amount of data to obtain a data set;
Assuming that an amount of data required to draw the true stress-true strain curve is X, and the total amount of data is Y, then the sampling interval is Δs=Y/X; conducting sampling at an interval of Δs rows to the data set [A₂,C₂], sequencing the sampled data, and denoting the data as [A₄,C₄], wherein A₄ is a data set of the force F, and C₄ is a data set of the engineering strain ε_{eng};
Mode III: sampling at fluctuation;
Assuming that a priority sampling number sequence is 0, 2, 5, 8, conducting prescreening to the data set [A₂,C₂], i.e., sampling the data of rows with the last numbers of 0, 2, 5, 8, denoting the total number of rows of the prescreened data as N, wherein N≥150, and denoting a generated data set as [A₅,C₅]; conducting data check to the first 70% of the data set [A₅,C₅];
u=C_{5w}-C_{5(w-1)}, and w=2, 3, . . . , 0.7N; conducting check row by row; when u<0, recording fluctuation once, adding one by one until the check is completed, and finally obtaining an accumulated fluctuation number v;
Assuming that a tolerance factor is k₁, and 0<k₁<0.05; when v<0.7*k₁*N, the screening is completed; otherwise, increasing the sampling period or adjusting the tolerance factor, and repeating the sampling and check process until requirements are met, wherein increasing the sampling period is to reduce the total number of data rows N by reducing the last numbers of the sampled rows;
Selecting a sampling mode to conduct data screening according to an actual working condition, sequencing the sampled data, and denoting the data as [A₆,C₆], wherein A₆ is a data set of the force F, and C₆ is a data set of the engineering strain ε_{eng}.

Further, the specific process of step (3) is as follows:
(3.1) Calculating the cross-sectional area S₀ of each range section:
Assuming that the circumferential tensile test sample has a diameter of D, a width of L₀, a thickness of δ, a milling width of P1 on one side of each range section, and a milling width of P2 on the other side of each range section;
The width of each range section is M=L₀−P1-P2;
The cross-sectional area of each range section is S₀=M·δ(L₀−P1−P2)·δ;
(3.2) Assuming that the data obtained by the tensile test are: the force F and the engineering strain ε_{eng};
An engineering stress σ_{eng} is:

$${σ}_{eng} = \frac{F}{2 {S}_{0}} = \frac{F}{2 δ ( {L}_{0} - P 1 - P 2 )} ;$$

The true stress σ is:

$$ σ = {σ}_{eng} ( 1 + {ε}_{eng} ) = \frac{F ( 1 + {ε}_{eng} )}{2 δ ( {L}_{0} - P 1 - P 2 )} ;$$

The true strain ε is: ε=ln(1+ε_{eng});
(3.3) Drawing the true stress-true strain curve.

Further, the process for obtaining a strength coefficient K and a strain hardening index β in a plastic section by calculation through a method of linear regression fitting is as follows: selecting the range of the plastic section on the true stress-true strain curve, and conducting linear regression calculation after logarithm calculation to obtain the strength coefficient K and the strain hardening index β:

$$σ = K {ε}^{β} ;$$

$$log σ = log K + β \cdot log ε .$$

The present invention has the following beneficial effects: the present invention proposes a method for obtaining circumferential mechanical property parameters of a pipe, which is suitable for large diameter pipes for finite element analysis. In the present invention, an engineering strain in a whole tension process of the test sample can be obtained in real time with a high accuracy based on a digital image correlation method by using a non-contact video extensometer. By applying the present invention, circumferential mechanical property data of a pipe can be accurately and comprehensively measured, true stress-strain data and curve can be obtained through a data processing algorithm, and a strain hardening behavior of a material can be more accurately described. By defining a range of a plastic section, a strength coefficient K and a strain hardening index β in the plastic section can be obtained by calculation, thus to provide accurate material data for finite element analysis of pipeline systems in military fields such as aviation, aerospace, engines, satellite and missiles as well as civilian fields such as automobiles, ships and high-speed railways.

### DESCRIPTION OF DRAWINGS

FIG. 1 is a design diagram of a flexible tensile test fixture.

FIG. 2 is a design diagram of a circumferential tensile test sample.

FIG. 3 shows calculation of a cross-sectional area of a range section.

FIG. 4 shows a true stress-true strain curve.

In the figures: 1—chuck; 2—pin; 3—D-shaped block; 4—range section.

### DETAILED DESCRIPTION

The embodiment proposes a method for obtaining circumferential mechanical property parameters of a pipe. A flexible tensile test fixture used for testing circumferential mechanical property parameters of a large diameter pipe is provided to conduct a tensile test on a circumferential tensile test sample of the pipe, and upward and downward pulling forces are provided by separating two D-shaped blocks 3 inside the flexible tensile test fixture to make the circumferential tensile test sample subjected to the pulling forces until fracture occurs. During the tensile test, it is necessary to obtain an interspeckle displacement in a whole tension process of the circumferential tensile test sample and a deformation condition of the circumferential tensile test sample in real time with a high accuracy based on a digital image correlation method by using a non-contact video extensometer, thus to obtain a deformation amount of the circumferential tensile test sample, further obtain an engineering strain of the circumferential tensile test sample, and then obtain time and force data of the tensile test in combination with a universal tensile testing machine. The deformation amount and the engineering strain of the circumferential tensile test sample as well as the time and the force of the tensile test are collectively referred to as tensile measurement data of the pipe. Preprocessing is conducted to the tensile measurement data of the pipe; the preprocessing means sequentially conducting: all abnormal data clearing before starting sampling, force check and convert, data cleaning and data screening; the preprocessed data includes only two types of data: the force and the engineering strain. Then true stress-true strain data of the circumferential tensile test sample of the pipe is obtained by calculation according to the preprocessed data, and a strength coefficient K and a strain hardening index β in a plastic section are obtained by calculation through a method of linear regression fitting by setting starting and ending strains of the plastic section.

In the present invention, a circumferential tensile test of the pipe is conducted based on the flexible tensile test fixture by using the circumferential tensile test sample with range sections 4 located on both sides, i.e., a tensile test method of a symmetrical test sample. The method avoids the influence of working hardening on a traditional transverse test sample to the greatest extent, and can avoid the influence of friction between the D-shaped blocks 3 of the flexible tensile test fixture and the range sections 4 to a certain extent. At the same time, the method is convenient for the non-contact video extensometer to acquire data.

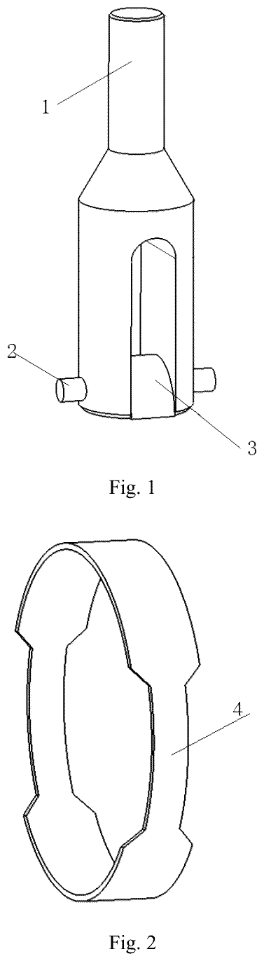

The flexible tensile test fixture comprises an upper fixture and a lower fixture, and each fixture comprises a chuck 1, a D-shaped block 3 and a pin 2, as shown in FIG. 1.

1) The chuck 1 comprises a clamping part, a gradual expansion part and a D-shaped block mounting part; the clamping part is a thin cylinder with a length of 60 mm and is used for matching with a V-shaped clamping block of the universal tensile testing machine, and a clamping force is ensured to be large enough by the two to prevent slipping. Upper and lower ends of the gradual expansion part are fixed with a bottom end of the clamping part and a top end of the D-shaped block mounting part, respectively. A D-shaped block mounting slot is formed in and penetrates through a side surface of the D-shaped block mounting part, a notch of the D-shaped block mounting slot has a length of 70 mm and a width of 20 mm and is suitable for the circumferential tensile test of a pipe with a diameter of 40-100 mm, and the length of the notch of the D-shaped block mounting slot shall be suitable for the tensile test of the circumferential tensile test sample of the pipe. The material of the chuck 1 is 40Cr subjected to tempering, so as to ensure that the part of the pin 2 will not be broken by a shearing force and improve the service life of the flexible tensile test fixture at the same time.

2) The circumferential tensile test sample is placed on an arc section of the D-shaped block 3; during the tensile test, the circumferential tensile test sample and the D-shaped block 3 are placed in the notch of the D-shaped block mounting slot; the pin 2 transversally penetrates through the D-shaped block mounting part of the chuck 1 and side surfaces of the D-shaped block 3 to connect the D-shaped block 3 with the D-shaped block mounting part of the chuck 1.

Further, the D-shaped block 3 is a flexible clamping element, and a diameter of the D-shaped block 3 is 1 mm-2 mm smaller than that of a circumferential tensile test sample to be measured for the convenience of clamping the circumferential tensile test sample. By designing D-shaped blocks 3 with different diameters, circumferential tension of circumferential tensile test samples of pipes with different diameters can be realized.

3) The pin 2 is used for connecting the D-shaped block mounting part of the chuck 1 with the D-shaped block 3, and the three are in clearance fit.

As shown in FIG. 2, in the embodiment, a circumferential tensile test sample of a certain type of pipe is taken as an example for a mechanical property test and parameter extraction, the circumferential tensile test sample has a symmetrical structure, the material of the circumferential tensile test sample is 1Gr18Ni10Ti, and it can be known from measurement that: the diameter D is 50 mm, and the thickness δ is 1.1 mm.

The specific implementation steps of the embodiment are as follows:

### Step I: Circumferential Tensile Test Sample Preparation:

Cutting a circumferential tensile test sample from a pipe to be measured by a laser cutting method; measuring that the width L₀ of the circumferential tensile test sample is 12.7 mm, P1 is 2.95 mm, and P2 is 3.12 mm; machining the range sections 4 symmetrically, and calculating a length L of each range section 4 of the circumferential tensile test sample as follows:

The length of each range section 4 is L=π·D/12=3.14*50/12=13 mm, and transition parts of the range sections 4 are smoothed;

Conducting speckle treatment to the range sections 4 of the circumferential tensile test sample, and spraying a white matte developer uniformly as a background to avoid interference with image recognition due to reflection of the circumferential tensile test sample, wherein the white developer shall be uniformly covered on the range sections 4 of the circumferential tensile test sample, no original color shall be exposed, and the white developer shall not be sprayed too thick, so as to avoid influence on recognition due to caving in a tension process. After natural air drying, spraying black matte paint spots as spots to form speckles, wherein the black matte paint spots shall be uniformly distributed in the form of small spots.

### Step II: Tensile Test Preparation:

Selecting D-shaped blocks 3 with a diameter of 48 mm-49 mm, and clamping the circumferential tensile test sample on the flexible tensile test fixture. The range sections 4 of the circumferential tensile test sample shall be placed on side surfaces to make the range sections 4 within a measuring range of the non-contact video extensometer. Adjusting the station of the non-contact video extensometer to ensure that the range sections 4 are completely within a field of view of a high-speed camera in the non-contact video extensometer, adjusting a focal length to complete range calibration, and enabling light source supplementary light and real-time calculation. Then clamping the flexible tensile test fixture of the pipe on the universal tensile testing machine, and using polytetrafluoroethylene to lubricate the contact positions of the D-shaped blocks 3 with the circumferential tensile test sample.

### Step III: Tensile Test:

Starting the universal tensile testing machine and the non-contact video extensometer to conduct the tensile test: completing relevant setup of the tensile test in software of the universal tensile testing machine, setting a cross beam displacement rate of the universal tensile testing machine to 5 mm/min, turning on a data acquisition function to obtain deformation data measured by the non-contact video extensometer, and starting the tensile test after force and displacement data of the universal tensile testing machine are cleared. Transmitting an image acquired by the high-speed camera in the non-contact video extensometer back to a computer, calculating a deformation amount of the circumferential tensile test sample by a digital image correlation method to further obtain a real-time engineering strain during the tensile test, obtaining time of the tensile test and a force of the tensile test by the universal tensile testing machine, exporting relevant test data after the test sample is broken and the tensile test is automatically stopped by the universal tensile testing machine, and ending the test. Part of the original data of the circumferential tensile test is shown in FIG. 4.

### Step IV: Test Data Preprocessing:

Preprocessing of circumferential mechanical property test data of the pipe is mainly to clean and screen the force data obtained by the universal tensile testing machine and the engineering strain data obtained by calculation through the non-contact video extensometer, thus to clear unreasonable values, sample and screen the data by a periodic sampling algorithm, and obtain a force and engineering strain data set.

TABLE 1
Part of original data of circumferential tensile test

| | B [symbol] | C [symbol] | |
|---|---|---|---|
| A [symbol] | Deformation | Engineering | D [symbol] |
| Force (kN) | amount (mm) | strain (mm/mm) | Time (s) |
| −0.63374 | 7.579771 | 0.1515954 | 0 |
| 0 | 0 | 0 | 0 |
| 7.462829 | 7.522981 | 0.1504596 | 152.4487 |
| 2.109 | 2277 | 45.54 | 2277 |
| 0.000948 | −0.01622 | −0.000324 | 0 |
| 0.01 | 342 | 6.84 | 0 |
| 7.462829 | 7.522981 | 0.1504596 | 152.4487 |
| 2.109 | 2277 | 45.54 | 2277 |
| 0 | 0 | 0 | 0 |
| 0 | 0 | 0 | 0 |
| 0.001 | −0.0074 | −0.00019 | 0.067 |
| 0.001 | −0.0036 | −0.00024 | 0.134 |
| 0.001 | 0 | 0.00000 | 0.201 |
| 0.001 | −0.00486 | −0.00032 | 0.27 |
| 0.001 | 0.00145 | 0.00010 | 0.337 |
| 0.001 | −0.00612 | −0.00041 | 0.406 |
| 0.001 | −0.00612 | −0.00041 | 0.474 |
| 0.001 | 0.00271 | 0.00018 | 0.541 |
| 0.001 | −0.00738 | −0.00049 | 0.608 |
| 0.001 | −0.0036 | −0.00024 | 0.708 |
| 0.001 | −0.00234 | −0.00016 | 0.775 |
| 0.001 | −0.0036 | −0.00024 | 0.842 |
| 0.001 | −0.01117 | −0.00074 | 0.909 |
| 0.001 | −0.00234 | −0.00016 | 0.976 |
| 0.001 | −0.00738 | −0.00049 | 1.043 |
| 0.001 | −0.00107 | −0.00007 | 1.111 |
| 0.002 | −0.00234 | −0.00016 | 1.178 |
| 0.001 | −0.00234 | −0.00016 | 1.245 |
| 0.001 | 0.00019 | 0.00001 | 1.312 |
| 0.001 | −0.00738 | −0.00049 | 1.379 |

[symbol]

indicates data missing or illegible when filed

An output data set of the universal tensile testing machine and the non-contact video extensometer includes the force (F), the deformation amount (ΔL), the engineering strain (ε_{eng}) and the time (t), as shown in Table 1. Reading a data column of the force F and denoting as column A₀=[A₀₁,A₀₂, . . . , A₀ₙ]^{T}=[−0.63374,0,7.462829, 2.109,0.000948, . . . ]^{T}, reading a data column of the deformation amount ΔL and denoting as column B₀=[B₀₁,B₀₂, . . . , B₀ₙ]^{T}=[7.579771,0,7.522981,2277,−0.01622, . . . ]^{T}, reading a data column of the engineering strain ε_{eng} and denoting as column C₀=[C₀₁,C₀₂, . . . , C₀ₙ]^{T}=[0.1515954,0,0.1504596,45.54,−0.000324, . . . ]^{T}, and reading a data column of the time t and denoting as column D₀=[D₀₁,D₀₂, . . . , D₀ₙ]^{T}=[0,0,152.4487,2277,0, . . . ]^{T}.

### (1) all Abnormal Data Clearing Before Starting Sampling:

Reading the column D₀, D₀₍ₚ₊₁₎−D₀ₚ=Tₚ, and p∈{1,2,3,4, . . . , n−1}; Tₚ represents a time difference between row p+1 and row p;

If

$$0 < {T}_{p} < k = \frac{1}{f}$$

(wherein 1.5<k<2, and f is a sampling frequency), data of row p+1 is marked as normal; otherwise, row p+1 and row p are marked as abnormal.

When 5 normal marks appears continuously, the reading is stopped, and all data in rows with abnormal marks is cleared. Data sets A₁=[0.001,0.001,0.001,0.001,0.001, . . . ]^{T}, B₁=[−0.00738,−0.0036,0,−0.00486,0.00145, . . . ]^{T} C₁=[−0.00049,−0.00024,0,−0.00032,0.0001, . . . ]^{T} and D₁=[0.067,0.134,0.201,0.27,0.337 . . . ]^{T} are obtained. The results are shown in Table 2.

TABLE 2
Part of data of circumferential tensile
test after abnormal data is cleared

| A1 | B1 | C1 | | |
|---|---|---|---|---|
| Force | Deformation | Engineering | D1 | A1′ = A1 |
| (kN) | amount (mm) | strain (mm/mm) | Time (s) | Force (N) |
| 0.001 | −0.0074 | −0.00049 | 0.067 | 1 |
| 0.001 | −0.0036 | −0.00024 | 0.134 | 1 |
| 0.001 | 0 | 0.00000 | 0.201 | 1 |
| 0.001 | −0.00486 | −0.00032 | 0.27 | 1 |
| 0.001 | 0.00145 | 0.00010 | 0.337 | 1 |
| 0.001 | −0.00612 | −0.00041 | 0.406 | 1 |
| 0.001 | −0.00612 | −0.00041 | 0.474 | 1 |
| 0.001 | 0.00271 | 0.00018 | 0.541 | 1 |
| 0.001 | −0.00738 | −0.00049 | 0.608 | 1 |
| 0.001 | −0.0036 | −0.00024 | 0.708 | 1 |
| 0.001 | −0.00234 | −0.00016 | 0.775 | 1 |
| 0.001 | −0.0036 | −0.00024 | 0.842 | 1 |
| 0.001 | −0.01117 | −0.00074 | 0.909 | 1 |
| 0.001 | −0.00234 | −0.00016 | 0.976 | 1 |
| 0.001 | −0.00738 | −0.00049 | 1.043 | 1 |
| 0.001 | −0.00107 | −0.00007 | 1.111 | 1 |
| 0.002 | −0.00234 | −0.00016 | 1.178 | 2 |
| 0.001 | −0.00234 | −0.00016 | 1.245 | 1 |
| 0.001 | 0.00019 | 0.00001 | 1.312 | 1 |
| 0.001 | −0.00738 | −0.00049 | 1.379 | 1 |

### (2) Force (F) Check and Convert:

Reading all data in column A₁:

[A₁₁,A₁₂, . . . , A₁ₘ]^{T}=[0.1,0.001,0.001,0.001,0.001, . . . ]^{T} and obtaining a maximum force Fmax=7.463.

If Fmax≤200, [A₁₁,A₁₂, . . . , A₁ₘ]^{T}×1000=[A′₁₁,A′₁₂, . . . , A′₁ₘ]^{T}=[1,1,1,1,1 . . . ]^{T} and [A₁₁,A₁₂, . . . , A₁ₘ]^{T} is overwritten with [A′₁₁,A′₁₂, . . . , A′₁ₘ]^{T} to obtain a new column A₁=[1,1,1,1,1 . . . ]^{T}. The results are shown in Table 2.

### (3) Data Cleaning:

Reading all data [C₁₁,C₁₂, . . . , C₁ₘ]^{T}=[−0.00049,−0.00024,0,−0.00032,0.0001, . . . ]^{T} in column C₁, and judging whether C_{1q}≤0, wherein q∈{1,2,3, . . . , m}.

Reading all data [C₁₁,C₁₂, . . . , C₁ₘ]^{T} in column C₁, and judging whether C_{1q}≤0, wherein q∈{1,2,3, . . . , m}; recording the last position where C_{1q} is zero or negative, clearing all data [A₁,B₁,C₁,D₁] before the position, deleting the data column of the deformation amount ΔL and the data column of the time t, and finally forming a new data set [A₂,C₂], wherein A₂=[231,231,232,233,234, . . . ]^{T}, and C₂=[0.00018,0.00035,0.00085,0.00043,0.00077, . . . ]^{T}. The results are shown in Table 3.

TABLE 3
Part of data of circumferential tensile test after data cleaning

| | C2 |
|---|---|
| A2 | Engineering |
| Force (N) | strain (mm/mm) |
| 231 | 0.00018 |
| 231 | 0.00035 |
| 232 | 0.00085 |
| 233 | 0.00043 |
| 234 | 0.00077 |
| 234 | 0.00069 |
| 236 | 0.00001 |
| 237 | 0.00077 |
| 238 | 0.00102 |
| 239 | 0.00085 |
| 240 | 0.00052 |
| 242 | 0.00111 |
| 243 | 0.00119 |
| 244 | 0.00094 |
| 247 | 0.00136 |
| 249 | 0.00052 |
| 250 | 0.00035 |
| 252 | 0.00085 |
| 252 | 0.00052 |
| 254 | 0.00043 |

### (4) Data Screening:

Conducting periodic sampling and data screening to the data set [A2,C2]. The present invention proposes 3 screening modes.

### Mode I: Sampling at Fixed Interval:

Specifying 10 as an interval for periodic sampling, conducting sampling at an interval of 10 rows to the data set [A₂,C₂], sequencing the sampled data, and denoting the data as [A₃,C₃], wherein A₃ is a data set of the force F, and C₃ is a data set of the engineering strain ε_{eng}. A₃=[231,240,256,278,300, . . . ]^{T}, and C₃=[0.00018,0.00052,0.00085,0.00052,0.00052 . . . ]^{T}. The results are shown in Table 4.

### Mode II: Sampling at Variable Interval:

Calculating a sampling interval according to a total amount of test data to obtain a data set.

Assuming that an amount of data required to draw the true stress-true strain curve is 150, and the total amount of data is 1742, then:

The sampling interval is Δs=1742/150=11.6; conducting sampling at an interval of 11 rows to the data set [A₂,C₂], sequencing the sampled data, and denoting the data as [A₄,C₄], wherein A₄ is a data set of the force F, and C₄ is a data set of the engineering strain ε_{eng}:

A₄=[231,242,260,285,312, . . . ]^{T}, and C₄=[0.00018,0.00111,0.00077,0.00094,0.00119, . . . ]^{T}. The results are shown in Table 4.

### Mode III: Sampling at Fluctuation:

Assuming that a priority sampling number sequence is 0, 2, 5, 8, conducting prescreening to the data set [A₂,C₂], i.e., sampling the data of rows with the last numbers of 0, 2, 5, 8, denoting the total number of rows of the prescreened data as N=697, and denoting a generated data set as [A₅,C₅]. A₅=[231,234,237,239,242, . . . ]^{T}, and C₅=[0.00035,0.00077,0.00077,0.00085,0.00111, . . . ]^{T}. Conducting data check to the first 70% of the data set [A₅,C₅]. The results are shown in Table 4.

TABLE 4
Part of data of obtained by 3 screening modes

| A3 | C3 | A4 | C4 | A5 | C5 |
|---|---|---|---|---|---|
| Force | Strain | Force | Strain | Force | Strain |
| (N) | (mm/mm) | (N) | (mm/mm) | (N) | (mm/mm) |
| 231 | 0.00018 | 231 | 0.00018 | 231 | 0.00035 |
| 240 | 0.00052 | 242 | 0.00111 | 234 | 0.00077 |
| 256 | 0.00085 | 260 | 0.00077 | 237 | 0.00077 |
| 278 | 0.00052 | 285 | 0.00094 | 239 | 0.00085 |
| 300 | 0.00052 | 312 | 0.00119 | 242 | 0.00111 |
| 329 | 0.00170 | 344 | 0.00186 | 247 | 0.00136 |
| 362 | 0.00170 | 385 | 0.00212 | 252 | 0.00085 |
| 398 | 0.00203 | 426 | 0.00220 | 254 | 0.00043 |
| 443 | 0.00220 | 492 | 0.00220 | 258 | 0.00136 |
| 504 | 0.00254 | 570 | 0.00245 | 266 | 0.00119 |

u=C_{5w}−C_{5(w-1)} (w=2, 3, . . . , 0.7N); conducting check row by row; when u<0, recording fluctuation once, adding one by one until the check is completed, and finally obtaining a fluctuation number v=48.

Assuming that a tolerance factor is k₁=0.04, so 0.7*k₁*N=0.7*0.04*697=19.52; as 48>19.52, it is necessary to increase the sampling period (reduce the last numbers of the sampled rows) or adjust the tolerance factor; after sampling for multiple times, it is finally determined that requirements for check can be met by sampling the data of rows with the last number of 2 and the tolerance factor k₁=0.04.

Selecting sampling mode III, sequencing the sampled data, and denoting the data as [A₆,C₆], wherein A₆ is a data set of the force F, and C₆ is a data set of the engineering strain ε_{eng}. A₆=[231,242,258,280,303, . . . ]^{T}, and C₆=[0.00035,0.00111,0.00136,0.00094,0.00102, . . . ]^{T}. The results are shown in Table 5.

TABLE 5

| A6 | C6 |
|---|---|
| Force (N) | Strain (mm/mm) |
| 231 | 0.00035 |
| 242 | 0.00111 |
| 258 | 0.00136 |
| 280 | 0.00094 |
| 303 | 0.00102 |
| 334 | 0.00119 |
| 370 | 0.00203 |
| 403 | 0.00186 |
| 454 | 0.00220 |
| 532 | 0.00262 |
| 612 | 0.00304 |
| 706 | 0.00296 |
| 819 | 0.00363 |
| 953 | 0.00380 |

### Step V: Property Parameter Obtaining:

Calculating the data of the force F and the engineering strain ε_{eng} obtained in step IV, calculating a true stress σ and a true strain ε, and outputting the true stress-true strain curve.

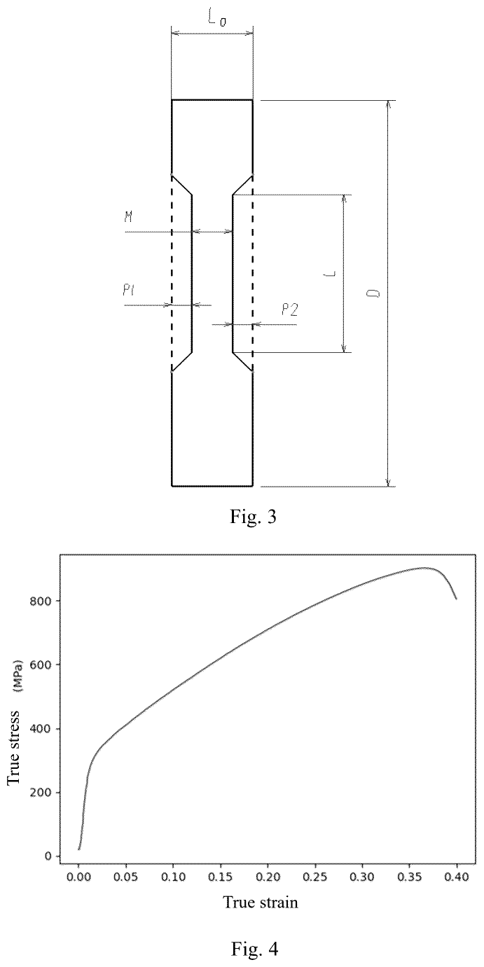

It is known that: as shown in FIG. 3, the diameter D of the circumferential tensile test sample of the pipe is 50 mm, and the thickness δ is 1.1 mm; after machining, the width L₀ of the circumferential tensile test sample is 12.7 mm, P1 is 2.95 mm, and P2 is 3.12 mm, then:

The width of each range section 4 is M=L₀−P1−P2=12.7−2.95−3.12=6.63 mm;

The cross-sectional area of each range section 4 is S₀=(L₀−P1−P2)·δ=6.63*1.1=7.293 mm;

An engineering stress σ_{eng} is obtained by calculation:

$${σ}_{eng} = \frac{F}{2 {S}_{0}} ;$$

The true stress σ is obtained by calculation:

$$σ = {σ}_{eng} ( 1 + {ε}_{eng} ) ;$$

The true strain ε is obtained by calculation: ε=ln(1+ε_{eng});

Inputting a range of the plastic section: a starting strain of the plastic section is 0.08, an ending strain of the plastic section is 0.2, and calculating a strength coefficient K and a strain hardening index β in the plastic section.

$$σ = K {ε}^{β} ;$$

$$log σ = log K + β \cdot log ε ;$$

Wherein σ is the true stress, and ε is the true strain.

It is obtained by calculation that the strength coefficient K is 1427, and the strain hardening index β is 0.4386.

1. A method for obtaining circumferential mechanical property parameters of a pipe, comprising the following steps:
step (1): adopting a flexible tensile test fixture used for testing circumferential mechanical property parameters of a pipe to conduct a tensile test on a circumferential tensile test sample of the pipe, and making the circumferential tensile test sample subjected to pulling forces until fracture occurs; during the tensile test, a deformation amount in a whole tension process of the circumferential tensile test sample is obtained in real time based on a digital image correlation method by using a non-contact video extensometer, thus to obtain an engineering strain of the circumferential tensile test sample, and then obtain time of the tensile test and a force of the tensile test in combination with a universal tensile testing machine; the deformation amount of the circumferential tensile test sample, the engineering strain of the circumferential tensile test sample, the time of the tensile test and the force of the tensile test are collectively referred to as tensile measurement data of the pipe;
step (2): preprocessing the tensile measurement data of the pipe; preprocessing means sequentially conducting: all abnormal data clearing before starting sampling, force check and convert, data cleaning and data screening; the preprocessed data includes only two types of data: the force of the tensile test and the engineering strain of the circumferential tensile test sample; the specific process is as follows:
using the tensile measurement data of the pipe as an output data set, including the force F, the deformation amount ΔL, the engineering strain ε_{eng} and the time t; reading a data column of the force F and denoting as column A₀=[A₀₁,A₀₂, . . . , A₀ₙ]^{T}, reading a data column of the deformation amount ΔL and denoting as column B₀=[B₀₁,B₀₂, . . . , B₀ₙ]^{T}, reading a data column of the engineering strain ε_{eng} and denoting as column C₀=[C₀₁,C₀₂, . . . , C₀ₙ]^{T}, and reading a data column of the time t and denoting as column D₀=[D₀₁,D₀₂, . . . , D₀ₙ]^{T};
(2.1) all abnormal data clearing before starting sampling;
reading the data column D₀, D₀₍ₚ₊₁₎−D₀ₚ=Tₚ, and p∈{1,2,3,4, . . . , n−1}; Tₚ represents a time difference between row p+1 and row p;
when
$$0 < {T}_{p} < k = \frac{1}{f} ,$$
data of row p+1 is marked as normal; otherwise, row p+1 and row p are marked as abnormal, wherein 1.5<k<2, and f is a sampling frequency;
When 5 normal marks appears continuously, the reading is stopped, and all data in rows with abnormal marks is cleared; obtaining a new data set: denoting a data column of the force F as column A₁=[A₁₁,A₁₂, . . . , A₁ₘ]^{T}, denoting a data column of the deformation amount ΔL as column B₁=[B₁₁,B₁₂, . . . , B₁ₘ]^{T}, denoting a data column of the engineering strain ε_{eng} as column C₁=[C₁₁,C₁₂, . . . , C₁ₘ]^{T}, and denoting a data column of the time t as column D=[D₁₁,D₁₂, . . . , D₁ₘ]^{T}; m<n;
(2.2) force check and convert;
reading all data [A₁₁,A₁₂, . . . , A₁ₘ]^{T} in column A₁, and obtaining a maximum force Fmax;
if Fmax≤200, [A₁₁,A₁₂, . . . , A₁ₘ]^{T}×1000=[A′₁₁,A′₁₂, . . . , A′₁ₘ]^{T}, and [A₁₁,A₁₂, . . . , A₁ₘ]^{T} is overwritten with [A′₁₁,A′₁₂, . . . , A′₁ₘ]^{T} to obtain a new column A₁;
if Fmax>200, a next step is conducted directly;
(2.3) data cleaning;
reading all data [C₁₁,C₁₂, . . . , C₁ₘ]^{T} in column C₁, and judging whether C_{1q}≤0, wherein q∈{1,2,3, . . . , m}; recording the last position where C_{1q} is zero or negative, clearing all data [A₁,B₁,C₁,D₁] before the position, deleting the data column of the deformation amount ΔL and the data column of the time t, and finally forming a new data set [A₂,C₂];
(2.4) data screening;
conducting periodic sampling and data screening to the data set [A₂,C₂] to reduce data and reduce a calculation amount on the premise of ensuring that a true stress-true strain curve can transition smoothly;
step (3): obtaining true stress-true strain data of the circumferential tensile test sample of the pipe by calculation according to the data obtained in step (2);
step (4): obtaining a strength coefficient K and a strain hardening index β in a plastic section by calculation through a method of linear regression fitting by setting starting and ending strains of the plastic section according to the true stress-true strain data of the circumferential tensile test sample; the strength coefficient K and the strain hardening index β in the plastic section as well as the true stress-true strain data of the circumferential tensile test sample are used for finite element analysis of a pipeline system.

2. The method for obtaining circumferential mechanical property parameters of a pipe according to claim 1, wherein the flexible tensile test fixture comprises an upper fixture and a lower fixture, and each fixture comprises a chuck (1), a D-shaped block (3) and a pin (2);
the chuck (1) comprises a clamping part, a gradual expansion part and a D-shaped block mounting part; the clamping part is a thin cylinder and is used for matching with a V-shaped clamping block of the universal tensile testing machine; upper and lower ends of the gradual expansion part are fixed with a bottom end of the clamping part and a top end of the D-shaped block mounting part, respectively; a D-shaped block mounting slot is formed in and penetrates through a side surface of the D-shaped block mounting part, and a length of a notch of the D-shaped block mounting slot is less than that of the circumferential tensile test sample; the circumferential tensile test sample is placed on an arc section of the D-shaped block (3); during the tensile test, the circumferential tensile test sample and the D-shaped block (3) are placed in the notch of the D-shaped block mounting slot; the pin (2) transversally penetrates through the D-shaped block mounting part of the chuck (1) and side surfaces of the D-shaped block (3) to connect the D-shaped block (3) with the D-shaped block mounting part of the chuck (1), and the D-shaped block mounting part of the chuck (1), the D-shaped block (3) and the pin (2) are in clearance fit.

3. The method for obtaining circumferential mechanical property parameters of a pipe according to claim 2, wherein the D-shaped block (3) is a flexible clamping element, and a diameter of the D-shaped block (3) is 1 mm-2 mm smaller than that of a circumferential tensile test sample to be measured; D-shaped blocks (3) with different diameters are designed to realize circumferential tension of circumferential tensile test samples of pipes with different diameters.

4. The method for obtaining circumferential mechanical property parameters of a pipe according to claim 1, wherein the circumferential tensile test sample is cut from a pipe to be measured, the circumferential tensile test sample has a symmetrical structure, and range sections (4) of the circumferential tensile test sample are located on both sides of a middle part of the circumferential tensile test sample.

5. The method for obtaining circumferential mechanical property parameters of a pipe according to claim 2, wherein it is assumed that the D-shaped block mounting slot has a length of B and a width of A, the pipe has a diameter of D, the circumferential tensile test sample has a width of L₀, then B>D/2+L₀, A is not less than L₀, thickness δ/diameter D of the pipe is less than 0.05, a length of each range section (4) of the circumferential tensile test sample is L=π·D/12, and transition parts of the range sections (4) are smoothed.

6. The method for obtaining circumferential mechanical property parameters of a pipe according to claim 2, wherein the tensile test comprises the following steps:
1) conducting speckle treatment to the range sections (4) of the circumferential tensile test sample: first, spraying a white matte developer uniformly, and spraying black matte paint spots after natural air drying to form speckles;
2) selecting matching D-shaped blocks (3) according to a size of the circumferential tensile test sample, and clamping the circumferential tensile test sample on the flexible tensile test fixture; then clamping the flexible tensile test fixture on the universal tensile testing machine, and using polytetrafluoroethylene to lubricate the contact positions of the D-shaped blocks (3) with the circumferential tensile test sample;
placing the range sections (4) after speckle treatment on side surfaces, making the range sections (4) within a measuring range of the non-contact video extensometer, setting a range to ensure that the range sections (4) are completely within a field of view of a high-speed camera of the non-contact video extensometer, adjusting a focal length and conducting range calibration;
3) starting the universal tensile testing machine and the non-contact video extensometer to conduct the tensile test, and obtaining the tensile measurement data of the pipe.

7. The method for obtaining circumferential mechanical property parameters of a pipe according to claim 1, wherein the data screening is conducted in mode I or mode II:
mode I: sampling at fixed interval;
specifying a single value as an interval for periodic sampling, conducting sampling at fixed interval to the data set [A₂,C₂], sequencing the sampled data, and denoting the data as [A₃,C₃], wherein A₃ is a data set of the force F, and C₃ is a data set of the engineering strain ε_{eng};
mode II: sampling at variable interval;
calculating a sampling interval according to a total amount of data to obtain a data set;
assuming that an amount of data required to draw the true stress-true strain curve is X, and the total amount of data is Y, then the sampling interval is Δs=Y/X; conducting sampling at an interval of Δs rows to the data set [A₂,C₂], sequencing the sampled data, and denoting the data as [A₄,C₄], wherein A₄ is a data set of the force F, and C₄ is a data set of the engineering strain ε_{eng}.

8. The method for obtaining circumferential mechanical property parameters of a pipe according to claim 1, wherein the data screening is sampling at fluctuation;
assuming that a priority sampling number sequence is 0, 2, 5, 8, conducting prescreening to the data set [A₂,C₂], i.e., sampling the data of rows with the last numbers of 0, 2, 5, 8, denoting the total number of rows of the prescreened data as N, wherein N≥150, and denoting a generated data set as [A₅,C₅]; conducting data check to the first 70% of the data set [A₅,C₅];
u=C_{5w}−C_{5(w-1)}, and w=2, 3, . . . , 0.7N; conducting check row by row; when u<0, recording fluctuation once, adding one by one until the check is completed, and finally obtaining an accumulated fluctuation number v;
assuming that a tolerance factor is k₁, and 0<k₁<0.05; when v<0.7*k₁*N, the screening is completed; otherwise, increasing the sampling period or adjusting the tolerance factor, and repeating the sampling and check process until requirements are met, wherein increasing the sampling period is to reduce the total number of data rows N by reducing the last numbers of the sampled rows;
selecting a sampling mode to conduct data screening according to an actual working condition, sequencing the sampled data, and denoting the data as [A₆,C₆], wherein A₆ is a data set of the force F, and C₆ is a data set of the engineering strain ε_{eng}.

9. The method for obtaining circumferential mechanical property parameters of a pipe according to claim 1, wherein the specific process of step (3) is as follows:
(3.1) calculating the cross-sectional area S₀ of each range section (4):
assuming that the circumferential tensile test sample has a diameter of D, a width of L₀, a thickness of δ, a milling width of P1 on one side of each range section (4), and a milling width of P2 on the other side of each range section (4);
the width of each range section (4) is M=L₀−P1−P2;
the cross-sectional area of each range section (4) is S₀=M·δ=(L₀−P1−P2)·δ;
(3.2) assuming that the data obtained by the tensile test are: the force F and the engineering strain ε_{eng};
the engineering strain σ_{eng} is:
$${σ}_{eng} = \frac{F}{2 {S}_{0}} = \frac{F}{2 δ ( {L}_{0} - P 1 - P 2 )} ;$$
the true stress σ is:
$$ σ = {σ}_{eng} ( 1 + {ε}_{eng} ) = \frac{F ( 1 + {ε}_{eng} )}{2 δ ( {L}_{0} - P 1 - P 2 )} ;$$
the true strain ε is: ε=ln(1+ε_{eng});
(3.3) drawing the true stress-true strain curve.

10. The method for obtaining circumferential mechanical property parameters of a pipe according to claim 1, wherein the process for obtaining a strength coefficient K and a strain hardening index β in a plastic section by calculation through a method of linear regression fitting is as follows: selecting the range of the plastic section on the true stress-true strain curve, and conducting linear regression calculation after logarithm calculation to obtain the strength coefficient K and the strain hardening index ε:
$$σ = K {ε}^{β} ;$$
$$log σ = log K + β \cdot log ε .$$
